(19) United States
(12) Patent Application Publication
(10) Pub. No.: US 2025/0270525 A1
(43) Pub. Date: Aug. 28, 2025

(54) METHODS AND COMPOSITIONS RELATING TO A GROUP II INTRON REVERSE TRANSCRIPTASE

(71) Applicant: BOARD OF REGENTS, THE UNIVERSITY OF TEXAS SYSTEM, Austin, TX (US)

(72) Inventors: Andrew ELLINGTON, Austin, TX (US); Inyup PAIK, Austin, TX (US); Sanchita BHADRA, Austin, TX (US); Shaunak KAR, Austin, TX (US)

(21) Appl. No.: 18/858,118

(22) PCT Filed: Apr. 17, 2023
(86) PCT No.: PCT/US2023/065843

§ 371 Date: Oct. 18, 2024

Related U.S. Application Data
(60) Provisional application No. 63/332,068, filed on Apr. 18, 2022.

(51) Int. Cl. C12N 9/12

(52) U.S. Cl. CPC C12N 9/1276; C07K 2319/85; C12Y 207/07049

(57) ABSTRACT

Disclosed herein are methods and compositions related to novel group II intron reverse transcriptases. These reverse transcriptases have been engineered to increase fidelity and/or processivity.

### RELATED APPLICATION

This application claims priority to, and the benefit of, U.S. Provisional Patent Application No. 63/332,068, filed Apr. 18, 2022, which is hereby incorporated by reference herein in its entirety.

### GOVERNMENT SUPPORT CLAUSE

This invention was made with government support under Grant No. R01 EB027202, awarded by the National Institutes of Health (NIH) and Grant No. NNX15AF46G, awarded by the National Aeronautics and Space Administration (NASA). The government has certain rights in the invention.

### SEQUENCE LISTING

The sequence listing submitted on Apr. 17, 2023, as an.XML file entitled “10046-459WO1_ST26_ST26.XML” created on Apr. 14, 2023, and having a file size of 17,827 bytes is hereby incorporated by reference pursuant to 37 C.F.R. § 1.52 (e) (5).

### BACKGROUND

Reverse transcription polymerase chain reaction, abbreviated as RT-PCR, is a well-known technique for amplifying RNA. In RT-PCR, an RNA strand is reverse transcribed intocomplementary DNA (cDNA), which is then amplified using DNA polymerase in the polymerase chain reaction. In the first step of this process, cDNA is made from an RNA template using deoxyribonucleotide phosphates and reverse transcriptase together with a DNA primer.

Synthesis of cDNA from the RNA template can be hindered by RNA secondary and tertiary structures, which consist of helices and various other kinds of kinks in the RNA strand. RNA secondary and tertiary structure can be decreased by carrying out the reaction at a higher temperature (e.g., above 50° C.) or by adding denaturing additives. However, the addition of denaturing additives is undesirable because it often reduces reverse transcriptase activity. Higher temperatures provide the advantage of increasing the specificity of DNA synthesis by decreasing non-specific primer binding. Unfortunately, only a limited number of reverse transcriptases, abbreviated RT, capable of operating at high temperature are currently available, and these exhibit relatively low fidelity DNA polymerization.

Given limitations of high fidelity RTs operable at high temperature, there is need in the art for improved RT compositions and methods to improve upon current RT-PCR techniques. The compositions and methods disclosed herein address these and other needs.

### SUMMARY

The present disclosure discloses a group II intron reverse transcriptase and methods of use thereof. In one aspect, disclosed herein is an amino acid sequence of a group II intron reverse transcriptase having at 90%, 95%, 99%, or 100% identity to any one of SEQ ID NOS: 2-5. In some embodiments, the amino acid sequence functions as a thermostable reverse transcriptase. In some embodiments, the thermostable reverse transcriptase is a group II intron reverse transcriptase. In some embodiments, the thermostable reverse transcriptase is a bacterial reverse transcriptase. In some embodiments, the bacterial reverse transcriptase is a Thermoanaerobacter siderophilus reverse transcriptase.

In one aspect, disclosed herein is a composition comprising an amino acid sequence, wherein said amino acid sequence comprises SEQ ID NO. 1 or a functional variant thereof, and a sequence with 90% or more identity to any of SEQ ID NOS: 10-13.

In some embodiments, the amino acid sequence further comprises a fusion peptide at the C-terminus of the linker. In some embodiments, the fusion peptide is a bacterial peptide. In some embodiments, the fusion amino acid sequence originates from a thermophilic bacterium.

In some embodiments, the thermophilic bacterium is Thermus thermophilus.

In some embodiments, the fusion peptide has 90% or more identity to any one of SEQ ID NOS: 6-9.

In one aspect, disclosed herein is a composition comprising an amino acid sequence, wherein said amino acid sequence comprises SEQ ID NO. 1 or a functional variant thereof, and a sequence with 90% or more identity to any one of SEQ ID NOS: 6-9, wherein SEQ ID NO: 1 and any one of SEQ ID NOS: 6-9 are separated by a linker.

In some embodiments, the linker is 5-40 amino acids in length. In some embodiments, the linker is rigid.

In one aspect, disclosed herein is a kit comprising an amino acid sequence with at least 90% identity to any one of SEQ ID NOS. 2-5 and reagents useful for nucleic acid amplification. In some embodiments, the kit comprises a reaction buffer solution. In some embodiments, the kit comprises a primer mix. In some embodiments, the kit comprises DEPC-treated or RNase-free water.

In one aspect, disclosed herein is a method of purifying a reverse transcriptase (RT) molecule, wherein the method comprises providing an RT molecule, wherein said RT molecule is associated with intron RNA, precipitating said RT molecule using a precipitation buffer with a high salt content, purifying said RT molecule, dialyzing collected polymerase fractions with high salt dialysis buffers and glycerol storage buffers, and collecting purified RT.

In some embodiments, said RT molecule has been produced by a transgenic cell. In some embodiments, said transgenic cell also produces other proteins. In some embodiments, said RT molecule comprises a fusion protein. In some embodiments, the precipitation buffer comprises ammonium sulfate, magnesium sulfate, sodium chloride, calcium chloride, potassium carbonate, or calcium sulfate.

In some embodiments, the first dialysis buffer comprises more than 100 mM salt. In some embodiments, the first dialysis buffer comprises more than 150 nM salt. In some embodiments, the first dialysis buffer comprises more than 200 nM salt. In some embodiments, the salt is sodium chloride, potassium chloride, potassium phosphate, or sodium bicarbonate. In some embodiments, the glycerol concentration is 35% or more. In some embodiments, the glycerol concentration is about 50% or more glycerol.

In some embodiments, said purification step uses heparin column purification. In some embodiments, the RT can be separated through a size exclusion column. In some embodiments, a high salt buffer is used in the size exclusion column. In some embodiments, the high salt buffer is sodium chloride. In some embodiments, sodium chloride is provided at 150 nM or higher.

In one aspect, disclosed herein is a method of amplifying RNA, the method comprising providing an RNA sequence to be amplified, exposing the RNA sequence to a composition comprising an amino acid sequence with at least 90% identity to any one of SEQ ID NOS: 2-5 and reagents needed for RNA amplification, and amplifying said RNA.

In some embodiments, the amplification method is RT-LAMP. In some embodiments, the amplification method is RT-PCR. In some embodiments, the RNA is a virus. In some embodiments, the virus is SARS-COV-2.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows Tsi Group II intron RT (RT12) purified in soluble form shown in SDS-PAGE.

FIGS. 2A and 2B show Tholoth LAMP-OSD assay detecting SARS-COV-2 viral RNA with Bst-LF and Bst2.0 DNA Polymerase amplifications. Both Bst-LF and Bst2.0 from NEB cannot detect 5000 copies of viral gRNA without a designated reverse transcriptase (black lines), however, can efficiently amplify viral gRNA in the presence of 0.1 pmol RT12 or RT12-3.

FIGS. 3A, 3B, 3C, and 3D show the fusion domain greatly improved processivity on Amplicon 1-3 with RT12-3 and Gs-5. Reverse transcription and quantitative PCR was performed using human RNA pol II coding mRNA (˜6 kb). Poly (A) primers were used for the reverse transcription and three sets of primers were selected near the 5′ end of the transcript for qPCR. X-axis: time (hh:mm:ss), Y-axis: fluorescence.

FIG. 4 shows the Purified Group II Intron Reverse Transcriptases are compatible with RT-qPCR assays. A standard Taqman qPCR assay for human GAPDH was performed using 10 ng of total human RNA as a template. All RT variants showed similar detection speeds with a widely used Superscript III (SSIII) reverse transcriptase in this assay. X-axis: time (hh:mm:ss), Y-axis: fluorescence.

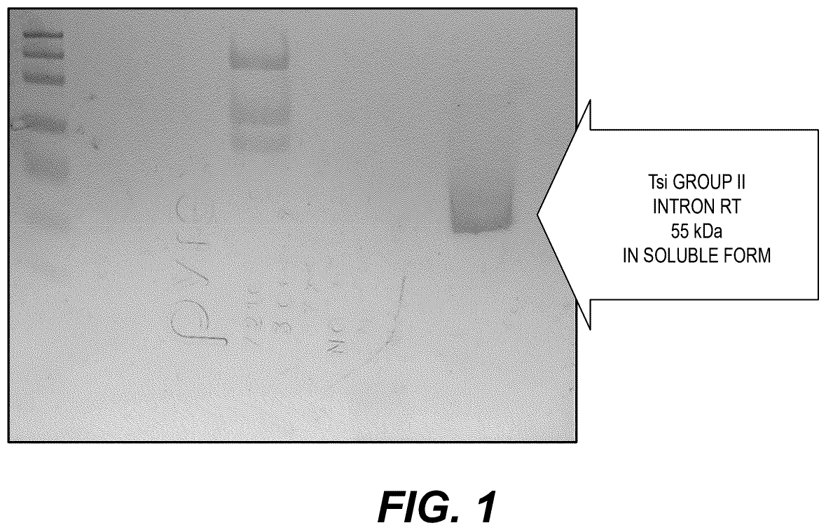

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

In this specification and in the claims which follow, reference will be made to a number of terms which shall be defined to have the following meanings:

Throughout the description and claims of this specification the word “comprises” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise.

“Thermophile/thermophilic” as used herein, refers to an organism that survives at relatively high temperatures, higher than 37° C. (100° F.).

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

As used herein, “codon” refers to the genetic code used by living cells to translate information encoded by genetic material (DNA or mRNA sequences of nucleotide triplets) into protein. This term also refers to the genetic code that specify which amino acids will be added next during protein synthesis.

“Expression” as used herein refers to the process by which information from a gene is used in the synthesis of a functional gene product that enables it to produce a peptide/protein end product, and ultimately affect a phenotype, as the final effect.

As used herein, “expression vector” refers to an expression construct, usually a plasmid or virus, designed to drive expression of a specific gene within a living cell. The vector is used to introduce a specific gene into a target cell and can utilize the cell's protein synthesis mechanisms to produce a desired protein product.

As used herein, “peptide” refers to a polymer of amino acids and does not imply a specific length of a polymer of amino acids. Thus, for example, the terms polypeptide, oligopeptide, protein, antibody, and enzyme are included within the definition of polypeptide. This term also includes peptides with post-expression modification, such as glycosylation (e.g., the addition of a saccharide), acetylation, phosphorylation, and the like. A “fusion protein” as used herein, refers to a protein having at least two heterologous polypeptides covalently linked in which one polypeptide comes from one protein sequence or domain and the other polypeptide comes from a second protein sequence or domain.

A “linker peptide” and “linker” as used herein, refers to a peptide used to link together two or more peptides.

As used herein, the term “polymerase” refers to an enzyme that synthesizes long chains of polymers or nucleic acids. DNA polymerase and RNA polymerase are used to assemble DNA and RNA molecules, respectively, by copying a DNA template strand using base-pairing interactions.

As used in the context of polymerase structure, “stabilized” refers to the structural integrity of the polymerase. As used in the context of polymerase function, “stabilized” refers to the polymerase ability to retain the function of synthesizing nucleic acid chains over time.

As used herein, the term “polymerase chain reaction” (“PCR”) refers to a method for increasing the concentration of a segment of a target sequence in a mixture of genomic DNA without cloning or purification. This process for amplifying the target sequence typically consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times to obtain a high concentration of an amplified segment of the desired target sequence. Unless otherwise noted, PCR, as used herein, also includes variants of PCR such as allele-specific PCR, asymmetric PCR, hot-start PCR, ligation-mediated PCR, multi-plex-PCR, reverse transcription PCR, or any of the other PCR variants known to those skilled in the art.

As used herein, the term “lysis” refers to the process of breaking down the membrane of a cell, often by viral, enzymatic, or osmotic mechanisms that compromise cellular integrity.

“Precipitate” or “pellet” as used herein refers to the solid formed from the process of transforming a dissolved substance into an insoluble solid once a solution becomes super-saturated with the substance.

“Supernatant” as used herein refers to the liquid remaining above the precipitated or solid substance following the process of precipitation.

As used herein, the term “variant” refers to a polypeptide derived from a parent protein by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’).

The term “thermostable,” as used herein, refers to the quality of a substance to resist irreversible change in its chemical and physical structure, often resisting decomposition or polymerization, at a relatively high temperature. In relation to the property of proteins, thermostable means to be resistant to changes in protein structure due to applied heat.

As used herein, the term “buffer” refers to a solution consisting of a mixture of acid and its conjugate base, or vice versa. The solution is used as a means of keeping the pH at a nearly constant range to be used in a wide variety of chemical and biological applications.

The term “transform,” as used herein, refers to the molecular biology application wherein genetic alteration of a cell resulting from the direct uptake and incorporation of an exogenous genetic material from its surroundings through the cell membrane.

“Culture” or “cell culture,” as used herein, refers to the process by which cells are grown under controlled conditions, generally outside their natural environment. After the cells of interest have been isolated from living tissue, they can subsequently be maintained under carefully controlled conditions. These conditions vary for each cell type, but generally consist of a suitable vessel with a substrate or medium that supplies the essential nutrients (amino acids, carbohydrates, vitamins, minerals), growth factors, hormones, and gases (CO₂, O₂), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture. “Cell culture” also refers to the culturing of cells derived from multicellular eukaryotes, especially animal cells, in contrast with other types of culture that also grow cells, such as plant tissue culture, fungal culture, and microbiological culture (of microbes).

As used herein, the term “dialysis” or “dialyze” refers to the process of separating molecules in solution by the difference in their rates of diffusion through a semipermeable membrane, such as dialysis tubing. Dialysis is a common laboratory technique for the removal of unwanted small molecules such as salts, reducing agents, or dyes from larger macromolecules such as proteins, DNA, or polysaccharides.

As used herein, the term “processivity” refers to the polymerase's ability to catalyze consecutive reactions without releasing the nucleic acid substrate. Processivity is the average number of nucleotides added by a polymerase enzyme per association event with the template strand. Because the binding of the polymerase to the template is the rate-limiting step in DNA synthesis, the overall rate of DNA replication is dependent on the processivity of the DNA polymerases performing the replication. DNA clamp proteins are integral components of the DNA replication machinery and serve to increase the processivity of their associated polymerases. Some polymerases add over 50,000 nucleotides to a growing DNA strand before dissociating from the template strand, giving a replication rate of up to 1,000 nucleotides per second.

“Amplicon,” as used herein, refers to a piece of DNA or RNA that is the source and/or product of amplification or replication events. These can be formed artificially, using various methods including PCR or ligase chain reactions (LCR), or naturally through gene duplication. It also refers to the common laboratory term, “PCR product.”

The term “transcript,” as used herein, refers to the single-stranded RNA product synthesized by transcription of DNA, and processed to yield various mature RNA products such as messenger RNA (mRNA), transfer RNA ((RNA), ribosomal RNA (rRNA), and guide RNA (gRNA).

### Stabilized Group II Intron Reverse Transcriptase Fusion Protein

Reverse transcriptases (RTs) are the enzymes essential for RT-PCR that can synthesize copies of cDNA from RNA templates. Retroviral RTs have been the only RTs used for biotechnology applications, including RT-PCR, next-generation RNA sequencing (RNA-seq), transcriptome and miRNA profiling, and RNA structure mapping. However, retroviral RTs have inherently low processivity and fidelity. For example, commercially available Avian Myeloblastosis Virus reverse transcriptase includes RNase H activity and can function at 37° C. but has a fidelity of only about 1.7×10⁻⁴. RNase H activity competes with the DNA polymerase activity and the primer binding site and, therefore, cDNA yield is lower. Other RT families, such as Group II intron RTs, have the ability to recognize and synthesize long continuous cDNA strands under high temperatures conditions. These RTs originate from mobile group II introns, which are genetic components that copy and paste themselves into different genomic locations by converting RNA back into DNA through reverse transcription. Although these RTs provide high fidelity and processivity of RNA to cDNA conversion, these RTs remain untapped as a source for biotechnology applications. Accordingly, there is a need for reverse transcriptase enzymes that can carry out reverse transcription at higher temperatures, including those that have high fidelity and processivity. Such enzymes are beneficial because higher temperatures decrease obstructing RNA secondary and tertiary structure and increase the specificity of reverse transcription by allowing the use of longer and more specific primers.

Disclosed herein is a stabilized group II intron reverse transcriptase fusion protein that includes a thermostable reverse transcriptase linked to a fusion peptide. Group II introns are found in bacteria, archaea, and the mitochondrial and chloroplast DNA of some eukaryotes. Group II introns encode a class of RNAs known for their self-splicing function. Under certain in vitro conditions, group II intron encoded RNAs can excise themselves from precursor mRNAs and ligate together their flanking exons, without the aid of a protein. Several group II introns also encode reverse transcriptases and are active mobile proteins. The group II intron reverse transcriptase assists RNA splicing by stabilizing the catalytically active RNA structure and then remains bound to the excised intron RNA in a ribonucleoprotein (RNP) that promotes intron mobility by the process termed “retrohoming.” Retrohoming occurs by a mechanism in which the excised intron RNA in the RNPs inserts directly into a DNA target site and is reverse transcribed by the reverse transcriptase. During retrohoming, the group II intron reverse transcriptase must produce an accurate cDNA copy of the intron RNA, which is typically 2-2.5 kilobases (kb) long and folds into highly stable, compact secondary and tertiary structures.

Disclosed herein are engineered group II intron-derived reverse transcriptases that exhibit relatively high fidelity and high processivity. The fidelity of these reverse transcriptases refers to the reliability of nucleotide incorporation during reverse transcription of RNA to DNA, with higher fidelity describing nucleotide copying with a low number of errors. The modified reverse transcriptases described herein can have higher fidelity than wild-type reverse transcriptases. In particular, the engineered reverse transcriptase described herein can have higher fidelity that the wild type sequence found in SEQ ID NO: 1. This fidelity can be increased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% compared to wild type, or can have 2, 3, 4, 5, 6, 7, 8, 9, or 10 times more fidelity, or any amount above, below, or in between these amounts.

The modified reverse transcriptases described herein can also have higher processivity than wild-type reverse transcriptases. In particular, the engineered reverse transcriptase described herein can have higher processivity that the wild type sequence found in SEQ ID NO: 1. This processivity can be increased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% compared to wild type, or can have 2, 3, 4, 5, 6, 7, 8, 9, or 10 times more processivity, or any amount above, below, or in between these amounts.

A wide variety of group II intron reverse transcriptases are known from a variety of organisms. In many embodiments, the group II intron reverse transcriptase can be a thermostable bacterial reverse transcriptase originating from Thermoanaerobacter siderophilus. Here, the group II intron reverse transcriptase with an amino acid sequence as set forth in SEQ ID NO: 1 encodes the original or parent group II intron reverse transcriptase without an attached linker or fusion peptide. Attaching a fusion peptide to a thermostable reverse transcriptase can provide one or more advantages. A stabilized reverse transcriptase fusion protein can: (a) increase stability at elevated temperatures; (b) promote higher processivity, (c) increase solubility, and/or (d) promote higher fidelity. In some embodiments, a reverse transcriptase of the invention may have a plurality of the properties listed above. In many embodiments, the fusion peptide is a small domain peptide found in the 30S ribosomal subunit THX from the thermophile bacterium Thermus thermophilus. The fusion peptide directly interacts with the target RNA being translated by the ribosome in a non-specific manner.

In many embodiments, the thermostable reverse transcriptase in connected to the fusion peptide via a linker peptide. Linker peptides are relatively short polypeptides used to attach one polymer molecule to another. Linker peptides can include from 1, 2, 3, 4, 5, 6, 7, 8, 9, 10, 11, 12, 13, 14, 15, 16, 17, 18, 19, 20, 21, 22, 23, 24, 25, 26,27, 28, 29, 30, 31, 32, 33, 34, 35, 36, 37, 38, 39, or 40 or more amino acids. In one embodiment, the linker peptide is a non-cleavable linker peptide. Non-cleavable refers to the inability of being dissociated or detached from the parent or fusion protein by a protease protein. While not intending to be bound by theory, linker peptides are believed to stabilize a fusion protein by decreasing the amount of movement of the fusion protein relative to the parent protein. Inclusion of a linker peptide between the fusion peptide and the thermostable reverse transcriptase can further enhance the listed advantages.

Examples of engineered reverse transcriptase molecules comprising a wild-type reverse transcriptase with a linker and a fusion peptide attached can be found in SEQ ID NOS: 2-5. These engineered reverse transcriptases can have an amino acid sequence with 80%, 81%, 82%, 83%, 84%, 85%, 86%, 87%, 88%, 89%, 90%, 91%, 92%, 93%, 94%, 95%, 96%, 97%, 98%, 99%, and 100% identity to SEQ ID NO: 2, 3, 4, or 5. Viewed in terms of number of amino acids that can vary, disclosed herein is a sequence comprising 1, 2, 3, 4, 5, 6, 7, 8, 9, 10, 11, 12, 13, 14, 15, 16, 17, 18, 19, 20, or more amino acids that vary compared to SEQ ID NO: 2-5. One of skill in the art will understand that the sequence can vary and still retain the function of SEQ ID NO: 2-5 to act as a thermostable reverse transcriptase. Therefore, contemplated herein are sequences which vary from SEQ ID NO: 2-5, but which retain their ability to act as thermostable reverse transcriptases (functional reverse transcriptases).

The group II intron reverse transcriptases typically comprise four functional domains: RT, the reverse transcriptase domain; X, the RNA splicing domain; D, a DNA-binding domain; and En, a DNA endonuclease domain that cleaves target DNA sequences. In some embodiments, the group II intron reverse transcriptases can have variance to one or more of these four domains. In some embodiments, the thermostable reverse transcriptase can have an amino acid sequence as set forth in SEQ ID NO: 1 (wild type reverse transcriptase), or a functional variant thereof, attached to an amino acid sequence with 80%, 81%, 82%, 83%, 84%, 85%, 86%, 87%, 88%, 89%, 90%, 91%, 92%, 93%, 94%, 95%, 96%, 97%, 98%, 99%, and 100% identity to any of SEQ ID NOS: 10-13. These are linker sequences, and it is understood that these linker sequences can be exchanged for each other in the reverse transcriptases described herein, so that any combination of linker and fusion peptide, as described herein, is contemplated. In some embodiments, one of the amino acid sequences as set forth by SEQ ID NOS: 10-13 is further attached to a bacterial fusion peptide. This can be at the C-terminal or N-terminal end. In a particular embodiment, it is at the C-terminal end. In some embodiments, the bacterial fusion peptide comprising the linkers disclosed herein can also have an amino acid sequence with 80%, 81%, 82%, 83%, 84%, 85%, 86%, 87%, 88%, 89%, 90%, 91%, 92%, 93%, 94%, 95%, 96%, 97%, 98%, 99%, and 100% identity to SEQ ID NO: 6-9. In some embodiments, the thermostable reverse transcriptase and the fusion peptide are separated by a linker peptide which is 5, 6, 7, 8. 9. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids in length.

In some embodiments, the thermostable reverse transcriptase can have an amino acid sequence as set forth in SEQ ID NO: 1, or a functional variant thereof, attached to an amino acid sequence with 80%, 81%, 82%, 83%, 84%, 85%, 86%, 87%, 88%, 89%, 90%, 91%, 92%, 93%, 94%, 95%, 96%, 97%, 98%, 99%, and 100% identity to any of SEQ ID NOS: 6-9. These are fusion peptides, and it is understood that these fusion peptides can be exchanged for each other in the reverse transcriptases described herein, so that any combination of linker and fusion peptide, as described herein, is contemplated. SEQ ID NOS: 6-9 represent fusion peptides and can be used along with SEQ ID NO: 1 or a variant thereof to create an engineered reverse transcriptase with properties which are superior to the wild-type sequence alone (SEQ ID NO: 1). The sequence for the reverse transcriptase and the sequence for the fusion peptide can be separated by a linker, for example. Any linker described herein can be used with the fusion peptides described herein.

### Purification and Use of Stabilized Group II Intron Reverse Transcriptase Fusion Protein

Also disclosed is a method for optimizing the purification protocols of group II intron reverse transcriptases. The group II intron reverse transcriptase can be purified in its soluble and active form. As used here, soluble refers to a substance that dissolves into a liquid to form a solution. The term active, as used herein, refers to the ability of a protein to carry out its intended functions. Purification of these reverse transcriptases is necessary to generate nucleic acid products from PCR with high fidelity and processivity, however group II intron reverse transcriptases have a propensity to aggregate into insoluble forms in vivo and in vitro. To date, purification of stable and soluble group II intron reverse transcriptases were not possible without an N-terminal maltose binding protein solubility tags. Thus, there remains a need for an optimized method to purify group II intron reverse transcriptases.

Example 1 gives a specific protocol which can be used to purify group II reverse transcriptases. It is understood that this is provided by way of example, and one of skill in the art would understand how to vary the protocol. However, the surprising and unexpected finding is that precipitation of these reverse transcriptases using “salting out” at a high salt concentration, and addition of glycerol during dialysis benefited the retention of transcriptase stability. The term “salting-out,” as used herein, refers to a biochemical purification technique that exploits the low solubility of certain macromolecules (i.e.: DNA or proteins) thus forcing them to no longer form a solution, and become a solid. Without the high salt concentrations and added glycerol, the results differ significantly, as can be seen in Example 2.

In some embodiments, the reverse transcriptase is precipitated using a precipitation buffer comprising a high salt content with at least 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 mM of salt. In some embodiments the precipitation buffer is used as a “salting-out” buffer comprising any one of the following salts: ammonium sulfate, magnesium sulfate, sodium chloride, calcium chloride, potassium carbonate, or calcium sulfate.

In some embodiments, the reverse transcriptase is dialyzed using a dialysis buffer comprising a high salt content with at least 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 mM of salt. In some embodiments, the first dialysis buffer comprises any one of the following salts: sodium chloride, potassium chloride, potassium phosphate, or sodium bicarbonate.

In some embodiments, a second dialysis step is performed. In some embodiments, the second dialysis buffer comprises 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% alcohol. In some embodiments, the alcohol is glycerol, propylene glycol, diethylene glycol, or ethylene glycol. When glycerol is used, it can be present at a concentration of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

In some embodiments, the purification method described herein uses a heparin column purification step. Heparin column protein purification exploits the electrostatic charges of proteins for removal from solutions. Heparin columns comprise negatively charged heparin proteins, which have high binding affinity the positive charges of other proteins. The binding affinity can be interrupted by exposure to different salt concentrations or acid to elute, or release, the protein fractions. Subsequently, the protein can be isolated and collected. In some embodiments, the purification method described herein uses a size exclusion column purification step. Size exclusion column purification separates proteins based on size by filtration through an immobilized gel matrix. The gel matrix is generally a fine, porous bead composition comprising dextran polymers, agarose, or polyacrylamide. These beads attract smaller proteins first, allowing for larger proteins to pass freely for collection. Collection of a desired protein depends on the bead selection within the immobilized gel matrix.

The present invention also provides a kit comprising the thermostable reverse transcriptase fusion protein and reagents useful for nucleic acid detection. In some embodiments, the kit comprises reagents and components including: (a) a primer mix, (b) a buffer solution, (c) a deoxynucleoside triphosphates (dNTPs) mix, (d) RNase-free or DEPC-treated water, and (e) a group II intron reverse transcriptase enzyme solution. In some embodiments, the kit components can be combined in the presence of RNA substrate for a reverse transcription-polymerase chain reaction (RT-PCR).

The present invention also provides a method for amplifying RNA and/or preparing cDNA, which is required for reverse transcription polymerase chain reaction (RT-PCR). In some embodiments, a target RNA sequence is exposed to the group II intron reverse transcriptase fusion protein and the reagents mentioned above. In some embodiments, the amplification method is reverse transcription loop mediated isothermal amplification (RT-LAMP) or RT-PCR. These reactions are performed within a temperature range where RNA secondary and tertiary structures are substantially decreased. This can be a temperature from about 45° C. to about 81° C. with a more preferred temperature range being from about 45° C. to about 65° C. Due to the high fidelity and other advantages of group II intron-derived RTs, their use may be preferred.

In some embodiments, the target RNA is a viral RNA. In some embodiments, the viral RNA originates from SARS-COV-2. Viral RNA is the genetical material of a virus to be translated into the protective outer protein shell or capsid. The SARS-COV-2 virus is a type of coronavirus, which are spherical shape with projections on the surface capsid, containing an RNA genome. This disease-causing virus spread rapidly in 2019 and 2020 leading to a worldwide pandemic causing respiratory illness and other secondary symptoms, later named COVID-19. Due to the quick spreading of SARS-COV-2, there is an increased need for more rapid and efficient detection of this virus in human populations. The present invention comprising improved methods for purifying a group II intron reverse transcriptase fusion protein also presented with novel findings of improved detection and processivity of the SARS-CoV-2 virus compared to a group II intron reverse transcription without a fusion protein.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1

### A. Background

Here, a novel group II intron reverse transcriptase (RT) has been engineered. The RT12 originated from Thermoanaerobacter siderophilus, a thermophilic bacteria isolated from hydrothermal vents in the Karymsky volcano on the Kamchatka peninsula. The RT12 was selected via rounds of high-temperature RT (65° C.) and qPCR selection. The amino acid sequence of the group II intron reverse transcriptase (sequence ID: WP_006569145) was back-translated and codon-optimized for the expression in E. coli and cloned into pKAR2 expression vector with carboxy terminal 6 histidine affinity tag (RT12). The RT was also engineered using a fusion domain library leading to the finding of 1j5E peptide fusion RT (RT12-3). The 1j5R peptide is a small domain found in 30S ribosomal subunit THX from an extreme thermophile Thermus thermophilus. Within the 30S ribosomal subunit, the 1j5R peptide was shown to directly interact with RNA that is being translated by the ribosome in a non-sequence specific manner. The fusion RT12-3 variant has SEQ ID NO:2. The parent RT12 has SEQ ID NO: 1.

### B. Optimized Purification Method

Group II intron Reverse transcriptases are well known for their propensity to aggregate in vivo and in vitro. The surface of the group II intron RTs is largely bound to co-expressed cognate intron RNA and thus scientists were not able to purify stable forms of the RTs without N-terminal MBP (maltose binding protein) solubility tags.

Optimization of the purification buffers led to successful purification of group II intron reverse transcriptase, RT12, in its soluble and active form.

The key steps include 8, 15,16,17. The key principle to stabilize group II intron RT is to keep either a high concentration of salt or glycerol in the buffers throughout the entire process.
1. The following protocol is detailed below. Transform pKAR2-RT12 and its variants into BL21 DE3 cells.
2. Seed culture transformed E. coli for o/n.
3. Main culture (500 mL to 2 L) and induction by 1 mM IPTG and 100 ng/ml aTc o/n at 18° C.
4. Resuspend in 30 mL Lysis buffer (50 mM Phosphate Buffer, pH 7.5, 300 mM NaCl, 0.1% Igepal CO-630, 5 mM MgSO4, 1× protease inhibitor cocktail).
5 Sonicate with 40% amplitude, 1 sec ON/4 sec off, for 4 min total sonication time.
6. Centrifuge at 35000 g for 30 min at 4° C.
7. Save the supernatant.
8. Precipitate total protein by ammonium sulfate salting-out method. (add 390 mg ammonium sulfate per milliliter of supernatant; 390 mg/ml)
9. Incubate at 4° C. for 30 minutes with gentle rotation.
10. Centrifuge at 35000 g for 30 min at 4° C.
11. Discard the supernatant and resuspend the pellet in 5 ml of Lysis buffer.
12. Filter resuspended lysate with 0.2 μm syringe filter.
13. Load lysate onto an FPLC machine and run heparin column purification.
14. Collect peaked fractions after the run. (RT12s typically fall into #40-45 fraction tubes)
15. Pool the fractions and dialyze into 2 L of High Salt Dialysis Buffer (40 mM Tris-HCl, pH 7.5, 200 mM NaCl, 1 mM DTT, 0.1% Igepal CO-630) twice (First 3-4 hours, Second O/N)
16. Optional: RT can be further separate through the size exclusion column using High Salt Size Exclusion buffer (50 mM Tris-HCl, pH 8.0, 200 mM NaCl, 0.1 mM EDTA, pH 8.0, 0.1% Igepal CO-630)
17. Directly transfer the dialysis cassette into 2 L of Final Dialysis Buffer (50% Glycerol, 50 mM Tris-HCl, pH 8.0, 50 mM KCl, 0.1% Tween-20, 0.1% Igepal CO-630) twice (First 3-4 hours, Second O/N)
   C. New RT is Compatible with LAMP Assay

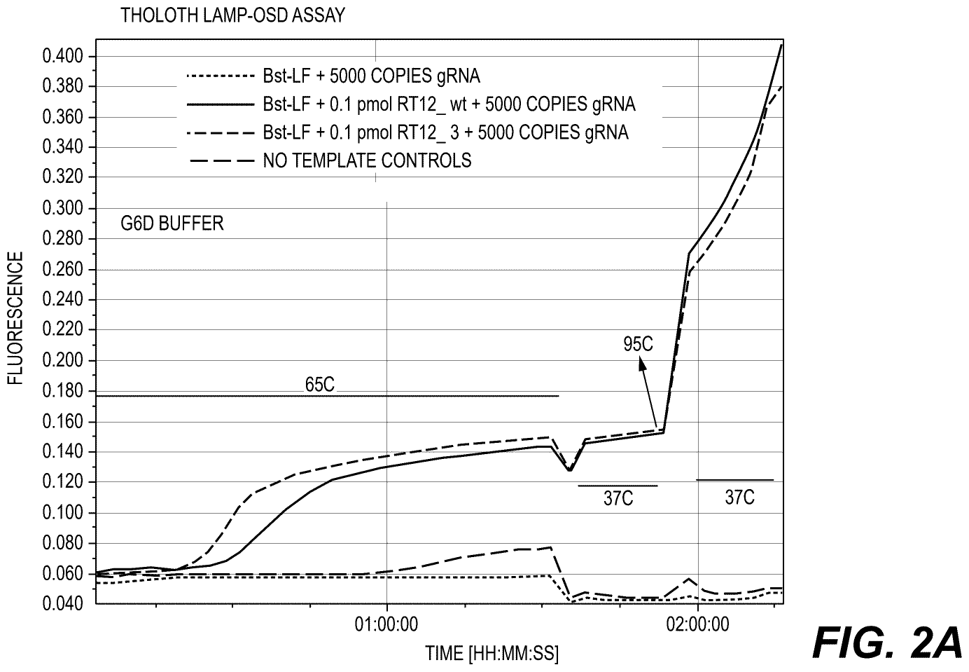

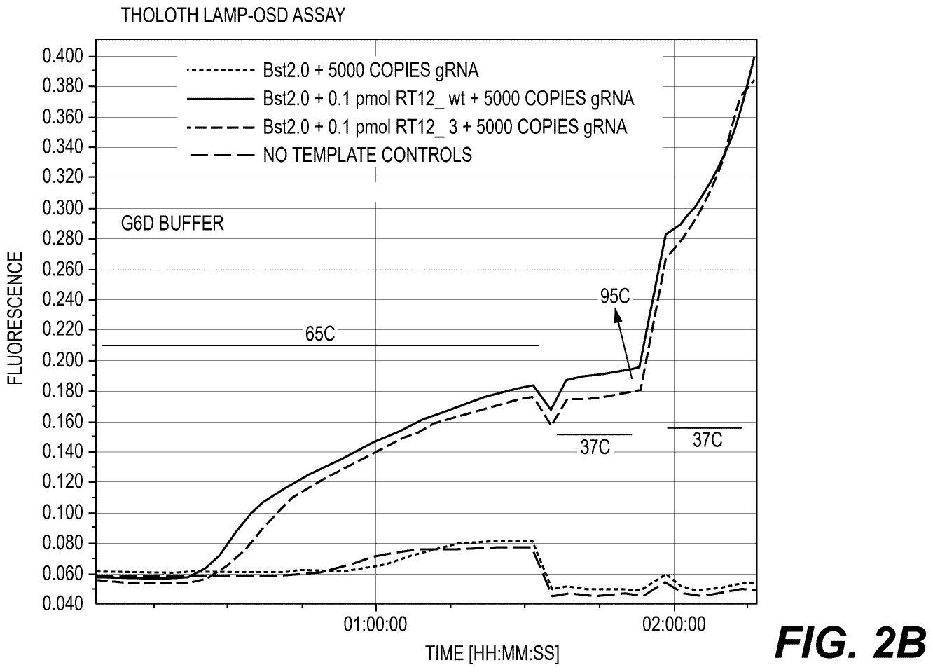

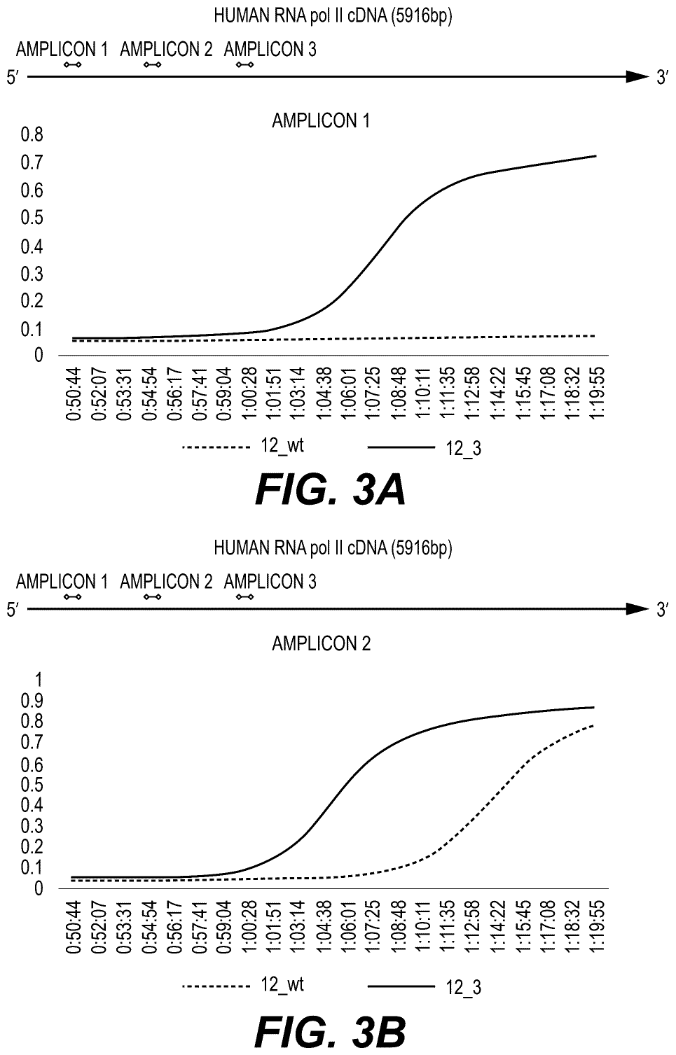

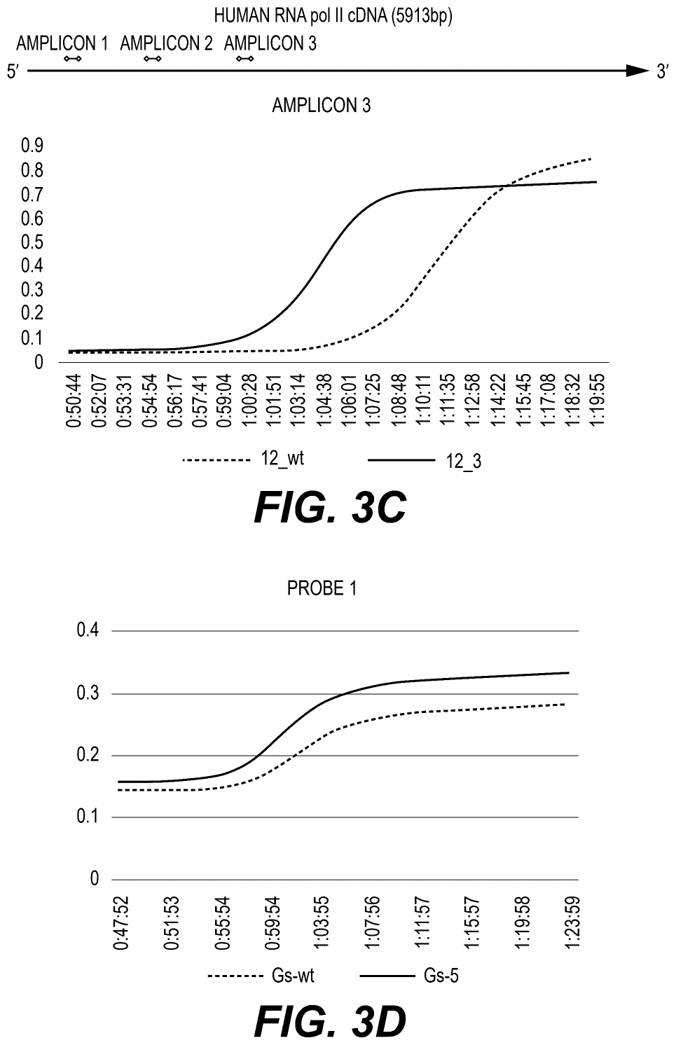

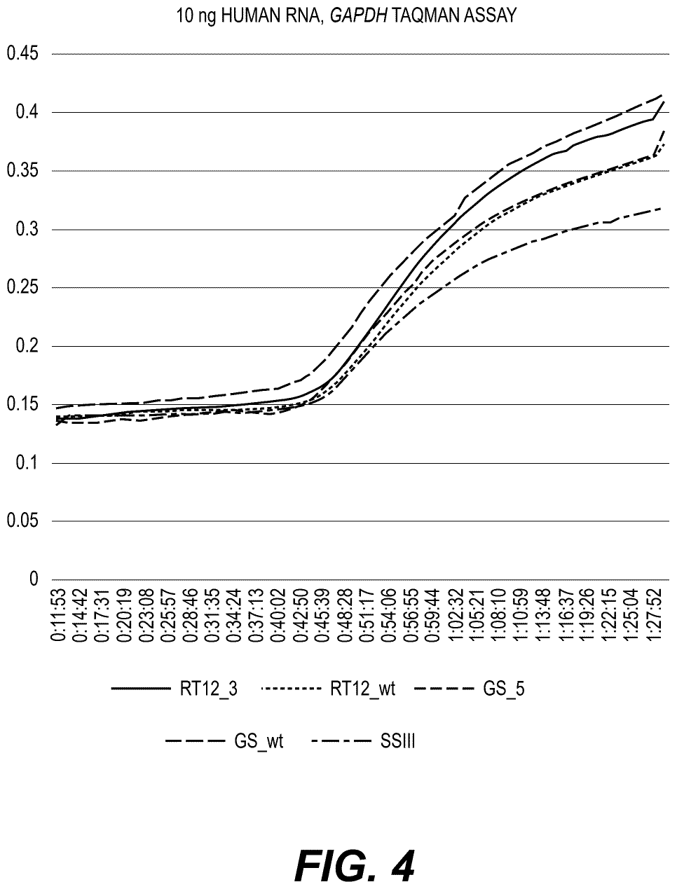

The compatibility of the purified RT12 and RT12-3 was examined in RT-LAMP-OSD assay (Tholoth LAMP-OSD) for SARS-COV-2 detection, which requires designated reverse transcriptase for the detection (FIG. 2). The results showed that the RT12 (red lines) and RT12-3 (blue lines) enabled the detection of 5000 copies of SARS-COV-2 viral gRNA, while Bst-LF and Bst2.0 alone (black lines) showed no amplification. In addition, the 1j5E peptide fusion domain greatly improved processivity of the reverse transcriptase. For instance, the RT12-3 showed much faster amplification of amplicons located near 5′ end of a long transcript; human RNA polymerase II in RT-qPCR assay (FIG. 3). This result shows that the RT12-3 was able to reverse transcribe the long transcript with high processivity starting from 3′ polyA tail to the 5′ end.

There are two key components that enabled solubilization of the group II intron reverse transcriptases These are:
1. High salt concentration in all intermediate dialysis buffers and
2. Glycerol in the final dialysis buffer

This combination allows the engineered reverse transcriptases to remain soluble in the buffers throughout the entire process.

### Example 2

Several failed attempts to purify soluble form of group II intron reverse transcriptases have been recorded. The first was using immobilized metal affinity chromatography (IMAC) (Ni-NTA): Sometimes called His-tag purification which is one of the most convenient and widely used protein purification methods. With the IMAC, group II intron reverse transcriptases could not be stabilized from E. coli.

Also, an unmodified Heparin column preparation method was attempted, which is a widely used method for polymerase purification. Using this method, the protein was not able to be stabilized.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A composition comprising an amino acid sequence with at least 90% identity to SEQ ID NO. 2, 3, 4, or 5.

2. (canceled)

3. (canceled)

4. (canceled)

5. The composition of claim 1, wherein the sequence functions as a thermostable reverse transcriptase.

6. The composition of claim 5, wherein the thermostable reverse transcriptase is a group II intron reverse transcriptase.

7. The composition of claim 5, wherein the thermostable reverse transcriptase is a bacterial reverse transcriptase.

8. The composition of claim 7, wherein the bacterial reverse transcriptase is a Thermoanaerobacter siderophilus reverse transcriptase.

9. A composition comprising an amino acid sequence, wherein said amino acid sequence comprises SEQ ID NO. 1 or a functional variant thereof, and a sequence with 90% or more identity to any of SEQ ID NOS: 10-13.

10. The composition of claim 9, wherein the amino acid sequence further comprises a fusion peptide at the C-terminus of the linker.

11. The composition of claim 10, wherein the fusion peptide is a bacterial peptide.

12. The composition of claim 10, wherein the fusion peptide originates from a thermophilic bacterium.

13. The composition of claim 12, wherein the thermophilic bacterium is Thermus thermophilus.

14. The composition of claim 10, wherein the fusion peptide has 90% or more identity to any one of SEQ ID NOS: 6-9.

15. A composition comprising an amino acid sequence, wherein said amino acid sequence comprises SEQ ID NO. 1 or a functional variant thereof, and a sequence with 90% or more identity to any one of SEQ ID NOS: 6-9, wherein SEQ ID NO: 1 and any one of SEQ ID NOS: 6-9 are separated by a linker.

16. The composition of claim 15, wherein the linker is 5-40 amino acids in length.

17. The composition of claim 15, wherein the linker is rigid.

18-42. (canceled)


SEQUENCE LISTING (15 sequences)

SEQ ID NO: 1 - protein, 478 aa, Thermoanaerobacter siderophilus
MDSKDMQRLQTTQQRGYPLNREMEFQKTTEVHSISSASEDGRNEVQRYTGKMLEMIVERGNMEAAYKRVVANKGSHGVDGMEVDELLPYLKENWPTIKQQLLEGKYKPQPVRRVEIPKPDGGVRLLGIPTALDRLIQQAIAQILNKVYNHTFSDSSYGFRPGRSAKDAIKAAEAYINEGYTWVVDMDLEKFFDRVNHDIIMSKLEKRIGDKRVLKLIRRYLESGVMINGIKISTEEGTPQGGPLSPLLANIMLDELDKELERRGHKFCRYADDCNIYVKSRSAGNRVMKSIKKFIESKLKLKVNEAKSAVDRPWRRKFLGFSFYTKENEVRIRIHEKSIKRFKEKVREITNRNKGISMENRIKRLNQITTGWVNYFGLADAKSIMKTLDEWIRRRLRACIWKQWKKIKTKHDNLVKLGVEEQKAWEYANTRKGYWRISNSPILNKTLTNKYFESIGYKSLSQRYLIVHNSGSHHHHHH

SEQ ID NO: 2 - protein, 519 aa, Thermoanaerobacter siderophilus
MDSKDMQRLQTTQQRGYPLNREMEFQKTTEVHSISSASEDGRNEVQRYTGKMLEMIVERGNMEAAYKRVVANKGSHGVDGMEVDELLPYLKENWPTIKQQLLEGKYKPQPVRRVEIPKPDGGVRLLGIPTALDRLIQQAIAQILNKVYNHTFSDSSYGFRPGRSAKDAIKAAEAYINEGYTWVVDMDLEKFFDRVNHDIIMSKLEKRIGDKRVLKLIRRYLESGVMINGIKISTEEGTPQGGPLSPLLANIMLDELDKELERRGHKFCRYADDCNIYVKSRSAGNRVMKSIKKFIESKLKLKVNEAKSAVDRPWRRKFLGFSFYTKENEVRIRIHEKSIKRFKEKVREITNRNKGISMENRIKRLNQITTGWVNYFGLADAKSIMKTLDEWIRRRLRACIWKQWKKIKTKHDNLVKLGVEEQKAWEYANTRKGYWRISNSPILNKTLTNKYFESIGYKSLSQRYLIVHNSGSAEAAAKEAAAKEAAAKAGSGKGDRRTRRGKIWRGTYGKYRPRKKKGS

SEQ ID NO: 3 - protein, 554 aa, Thermoanaerobacter siderophilus
MDSKDMQRLQTTQQRGYPLNREMEFQKTTEVHSISSASEDGRNEVQRYTGKMLEMIVERGNMEAAYKRVVANKGSHGVDGMEVDELLPYLKENWPTIKQQLLEGKYKPQPVRRVEIPKPDGGVRLLGIPTALDRLIQQAIAQILNKVYNHTFSDSSYGFRPGRSAKDAIKAAEAYINEGYTWVVDMDLEKFFDRVNHDIIMSKLEKRIGDKRVLKLIRRYLESGVMINGIKISTEEGTPQGGPLSPLLANIMLDELDKELERRGHKFCRYADDCNIYVKSRSAGNRVMKSIKKFIESKLKLKVNEAKSAVDRPWRRKFLGFSFYTKENEVRIRIHEKSIKRFKEKVREITNRNKGISMENRIKRLNQITTGWVNYFGLADAKSIMKTLDEWIRRRLRACIWKQWKKIKTKHDNLVKLGVEEQKAWEYANTRKGYWRISNSPILNKTLTNKYFESIGYKSLSQRYLIVHNSGSGGGGSGGGGSGGGGSGSGMATVKFKYKGEEKEVDISKIKKVWRVGKMISFTYDEGGGKTGRGAVSEKDAPKELLQMLEKQKK

SEQ ID NO: 4 - protein, 556 aa, Thermoanaerobacter siderophilus
MDSKDMQRLQTTQQRGYPLNREMEFQKTTEVHSISSASEDGRNEVQRYTGKMLEMIVERGNMEAAYKRVVANKGSHGVDGMEVDELLPYLKENWPTIKQQLLEGKYKPQPVRRVEIPKPDGGVRLLGIPTALDRLIQQAIAQILNKVYNHTFSDSSYGFRPGRSAKDAIKAAEAYINEGYTWVVDMDLEKFFDRVNHDIIMSKLEKRIGDKRVLKLIRRYLESGVMINGIKISTEEGTPQGGPLSPLLANIMLDELDKELERRGHKFCRYADDCNIYVKSRSAGNRVMKSIKKFIESKLKLKVNEAKSAVDRPWRRKFLGFSFYTKENEVRIRIHEKSIKRFKEKVREITNRNKGISMENRIKRLNQITTGWVNYFGLADAKSIMKTLDEWIRRRLRACIWKQWKKIKTKHDNLVKLGVEEQKAWEYANTRKGYWRISNSPILNKTLTNKYFESIGYKSLSQRYLIVHNSGSGGGGSGGGGSGGGGSGSGMVKVKFKYKGEEKEVDTSKIKKVWRVGKMVSFTYDDNGKTGRGAVSEKDAPKELLDMLARAEREKK

SEQ ID NO: 5 - protein, 550 aa, Thermoanaerobacter siderophilus
MDSKDMQRLQTTQQRGYPLNREMEFQKTTEVHSISSASEDGRNEVQRYTGKMLEMIVERGNMEAAYKRVVANKGSHGVDGMEVDELLPYLKENWPTIKQQLLEGKYKPQPVRRVEIPKPDGGVRLLGIPTALDRLIQQAIAQILNKVYNHTFSDSSYGFRPGRSAKDAIKAAEAYINEGYTWVVDMDLEKFFDRVNHDIIMSKLEKRIGDKRVLKLIRRYLESGVMINGIKISTEEGTPQGGPLSPLLANIMLDELDKELERRGHKFCRYADDCNIYVKSRSAGNRVMKSIKKFIESKLKLKVNEAKSAVDRPWRRKFLGFSFYTKENEVRIRIHEKSIKRFKEKVREITNRNKGISMENRIKRLNQITTGWVNYFGLADAKSIMKTLDEWIRRRLRACIWKQWKKIKTKHDNLVKLGVEEQKAWEYANTRKGYWRISNSPILNKTLTNKYFESIGYKSLSQRYLIVHNSGSGGGGSGGGGSGGGGSGSGGKYFRHKLPDDYPIGSGSMKPVKVKTPAGKEAELVPEKVWALAPKGRKGVKIGLFKDPET

SEQ ID NO: 6 - protein, 27 aa, Thermoanaerobacter siderophilus
KGDRRTRRGKIWRGTYGKYRPRKKKGS

SEQ ID NO: 7 - protein, 64 aa, Thermoanaerobacter siderophilus
MATVKFKYKGEEKEVDISKIKKVWRVGKMISFTYDEGGGKTGRGAVSEKDAPKELLQMLEKQKK

SEQ ID NO: 8 - protein, 84 aa, Thermoanaerobacter siderophilus
GGGGSGGGGSGGGGSGSGMVKVKFKYKGEEKEVDTSKIKKVWRVGKMVSFTYDDNGKTGRGAVSEKDAPKELLDMLARAEREKK

SEQ ID NO: 9 - protein, 60 aa, Thermoanaerobacter siderophilus
GKYFRHKLPDDYPIGSGSMKPVKVKTPAGKEAELVPEKVWALAPKGRKGVKIGLFKDPET

SEQ ID NO: 10 - protein, 20 aa, Thermoanaerobacter siderophilus
AEAAAKEAAAKEAAAKAGSG

SEQ ID NO: 11 - protein, 18 aa, Thermoanaerobacter siderophilus
GGGGSGGGGSGGGGSGSG

SEQ ID NO: 12 - protein, 9 aa, Thermoanaerobacter siderophilus
GSGSGSGSG

SEQ ID NO: 13 - protein, 15 aa, Thermoanaerobacter siderophilus
AEAAAKEAAAKAGSG

SEQ ID NO: 14 - protein, 428 aa, Geobacillus stearothermophilus
MALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQGGSHHHHHH

SEQ ID NO: 15 - protein, 464 aa, Geobacillus stearothermophilus
MALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQGGSAEAAAKEAAAKAGSGKGDRRTRRGKIWRGTYGKYRPRKKKGS